(19) United States
(12) Patent Application Publication
(10) Pub. No.: US 2018/0265472 A1
(43) Pub. Date: Sep. 20, 2018

(54) PROCESS FOR PREPARING ARIPIPRAZOLE LAUROXIL AND INTERMEDIATES THEREOF

(71) Applicant: ScinoPharm Taiwan, Ltd., Shan-Hua (TW)

(72) Inventors: Tsung-Yu Hsiao, Shan-Hua (TW); Yu-Hui Huang, Shan-Hua (TW)

(73) Assignee: ScinoPharm Taiwan, Ltd.

(21) Appl. No.: 15/915,956

(22) Filed: Mar. 8, 2018

Related U.S. Application Data
(60) Provisional application No. 62/472,693, filed on Mar. 17, 2017.

(51) Int. Cl. C07D 215/22

(52) U.S. Cl. CPC C07D 215/22

(57) ABSTRACT

A process for preparing a compound of formula I′:

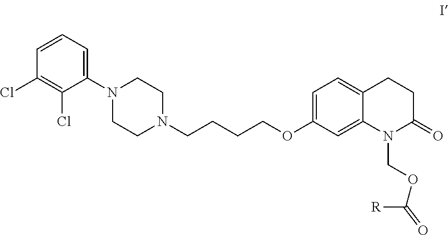

or a pharmaceutically acceptable salt thereof, in which R represents optionally substituted or unsubstituted alkyl group, comprising reacting a compound of formula III:

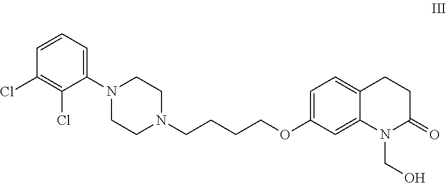 [SMILES: O=C1CCc2ccc(OCCCCN3CCN(c4cccc(Cl)c4Cl)CC3)cc2N1CO]

with a carboxylic acid in the presence of a coupling reagent and an organic solvent to obtain the compound of formula I′.

### CROSS-REFERENCES TO RELATED APPLICATIONS

This application claims priority from U.S. Provisional Patent Application Ser. No. 62/472,693, which was filed on Mar. 17, 2017. The entire content of this provisional application is incorporated herein by reference.

### BACKGROUND OF THE INVENTION

Aripiprazole lauroxil (formerly known as N-lauroyloxymethyl aripiprazole) is a novel lipid ester prodrug of aripiprazole for the treatment of schizophrenia in adults, which is a chronic, severe and disabling brain disorder. Aripiprazole lauroxil was developed and designed by Alkermes, Inc., and was approved by FDA in 2015 under the trade name Aristada™. Aristada™ is available as a white to off-white sterile aqueous extended-release suspension for intramuscular injection with different doses of aripiprazole lauroxil, including 441 mg, 662 mg and 882 mg. Following intramuscular injection, Aristada™ is likely converted by enzyme-mediated hydrolysis to N-hydroxym ethyl aripiprazole (active moiety), which is then hydrolyzed to aripiprazole. Aripiprazole is an orally administered small-molecule compound which is commercially available under the trade name Abilify® owned by Otsuka Therapeutics, Inc. for the treatment of a number of CNS disorders. Aristada™ is similar to Abilify® in effect of typical antipsychotic drugs but allows for dosing flexibility. Depending on each individual patient's needs, treatment with Aristada™ can be initiated at a dose of 441 mg, 662 mg or 882 mg administered monthly, which corresponds to 300 mg, 450 mg and 600 mg of aripiprazole respectively or dose of 882 mg every six weeks. Besides, elderly patients with dementia-related psychosis treated with antipsychotic drugs are at an increased risk of death. Aristada™ is not approved for the treatment of elderly patients with dementia-related psychosis.

In U.S. Patent Application Publication No. 20150274670A1, a mixture of aripiprazole, triethylamine, 37% aqueous solution of formaldehyde and N, N-dimethylformamide was heated to 80° C., leading to a mixture of hemi-aminal compound M1 and aripiprazole. A mixture was isolated as a white solid in 87.2% yield containing 21.8% of aripiprazole and 65.4% of desired hemi-aminal compound, M1. The M1 mixture was converted to the prodrug, aripiprazole lauroxil, in the presence of dodecanoic anhydride under basic condition. The desired product was isolated in 21% yield as a crystalline solid. The overall yield from aripiprazole to aripiprazole lauroxil was about 13.7%. See Scheme 1, path A below.

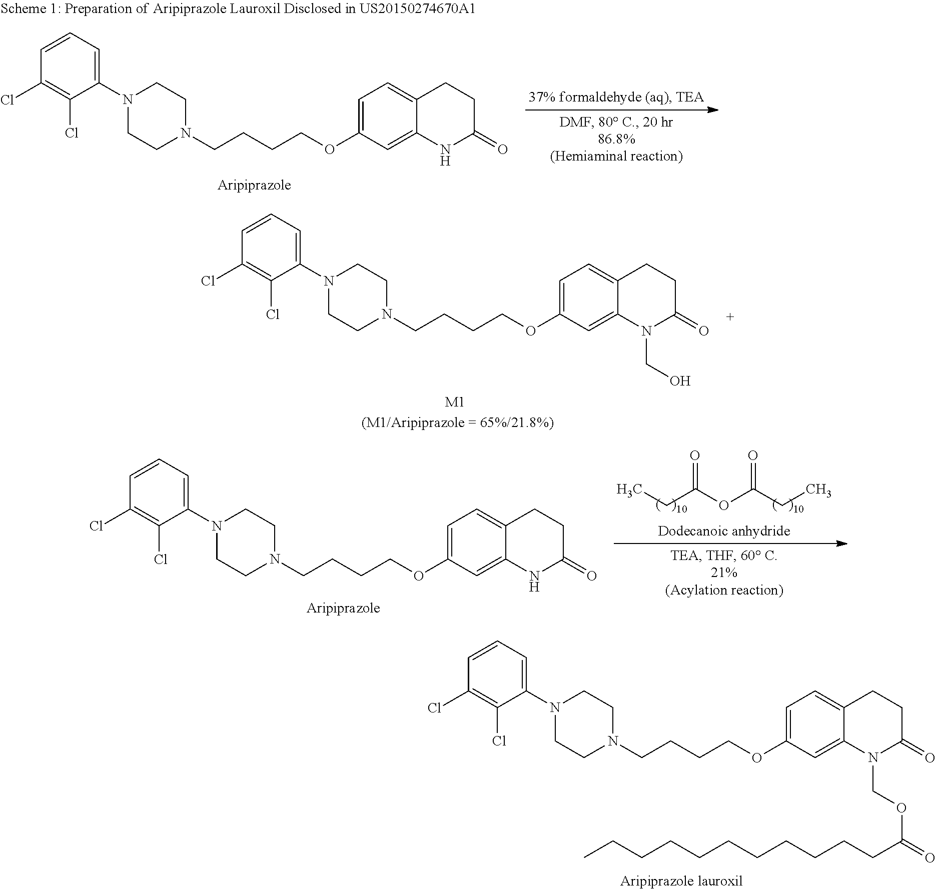

The same synthetic approach is also revealed in other patent applications, such as International Patent Application Publication Nos. WO2011140183A1, WO2015143145A1, and WO2012129156A1, and U.S. Patent Application Publication No. 2014-088115A1.

U.S. Patent Application Publication No. 20150274670A1 reports another approach for synthesis in which aripiprazole is coupled with chloromethyl ester at 90° C. under strong basic condition with a catalytic amount of NaI providing compound I with 70% yield. The resultant chloromethyl ester is prepared from acyl chloride reacting with paraformaldehyde in the presence of a catalyst, ZnCl₂. Similarly, ziprasidone free base as described in this patent application is coupled in a similar manner but under milder basic condition with a different catalyst, 4-DMAP, leading to compound II with 27% yield. As described in this patent application, synthesis of aripiprazole lauroxil can be accomplished in a similar manner by using chloromethyl dodecanoate. See Scheme 2, path B below.

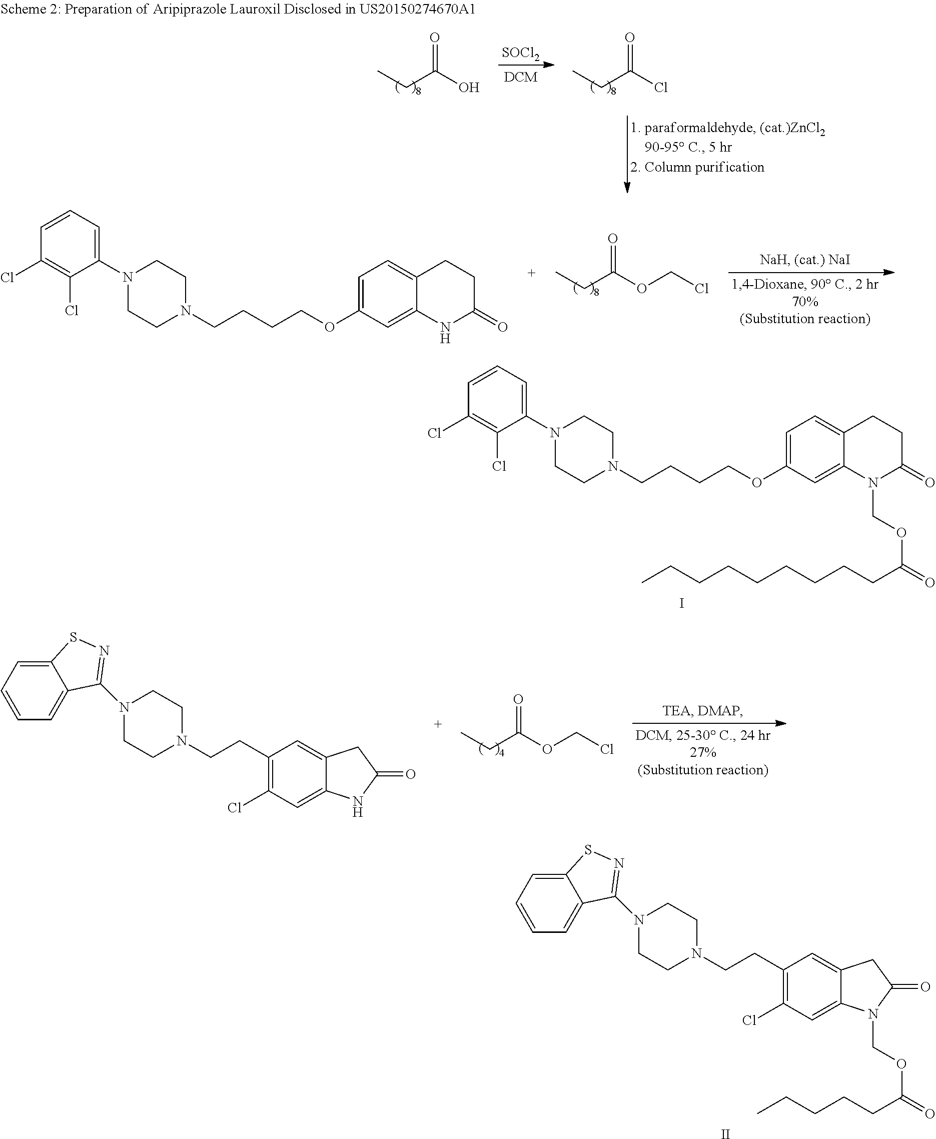

International Patent Application Publication No. WO2011140183A1 discloses that a suspension of M1 and aripiprazole mixture in DCM is reacted with butyryl chloride under basic condition, leading to compound III with 95% yield. As described in this patent application, synthesis of aripiprazole lauroxil can be accomplished in a similar manner by using dodecanoyl chloride. See Scheme 3, path C below.

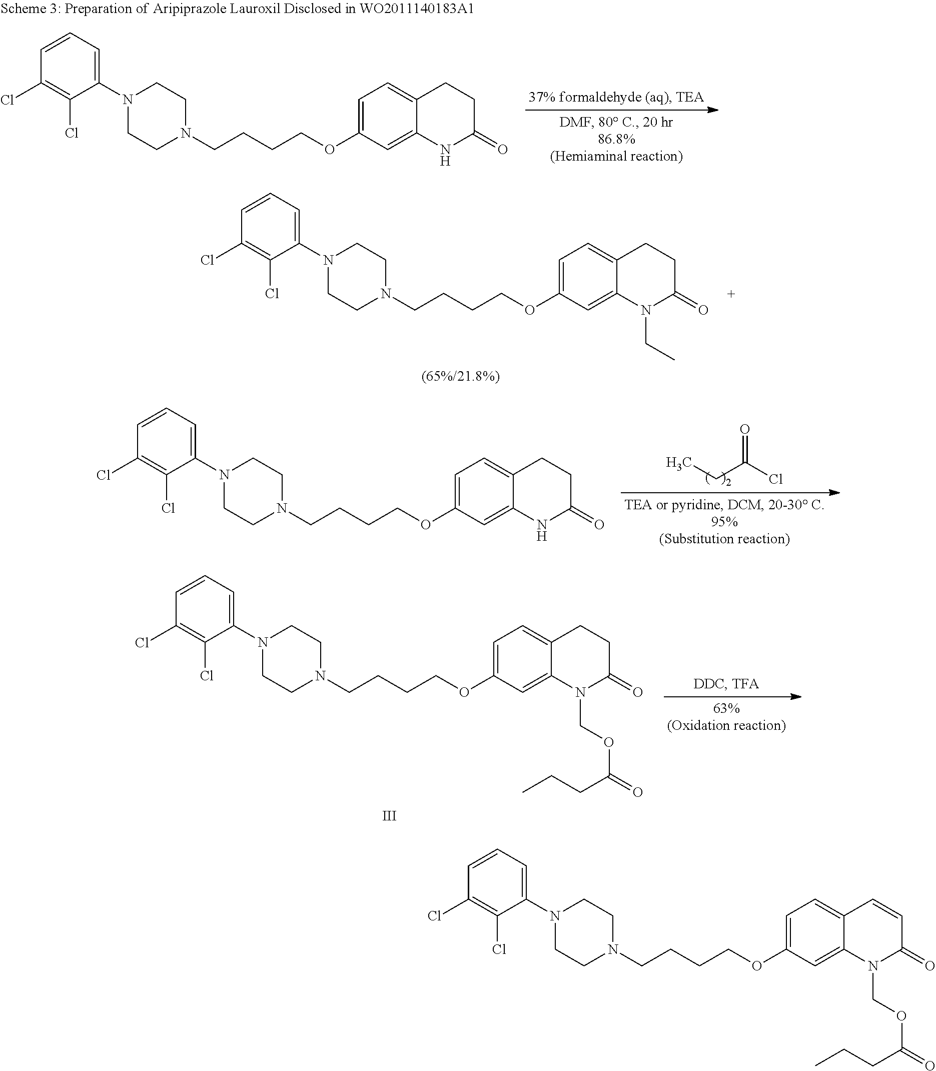

U.S. Patent Application Publication No. 20160051546A1 discloses that aripiprazole lauroxil is crystallized from two solvent systems to control its particle size distribution and surface area. In this patent application, crystallization of aripiprazole lauroxil is achieved in IPAc-n-heptane systems. The resultant prodrug could be dissolved in IPAc at 55-65° C. and hot heptane is added at this temperature. While cooling the mixture to a supersaturated condition (at about 34° C.), crystallization occurs. When the temperature of the mixture is within the range of about 0-5° C., above 33.8-34° C., the mixture is homogenized to form crystallized particles of aripiprazole lauroxil having a surface area of about 0.5 to about 3.3 m²/g. Control of the solution temperature to target a specific onset temperature for crystallization is important to control the final particle size distribution and surface area of the aripiprazole lauroxil crystals.

In view of the above, there remains a need for the development of improved processes for the preparation of aripiprazole lauroxil.

### SUMMARY OF THE INVENTION

The present application provides a process for preparing a compound of formula I′:

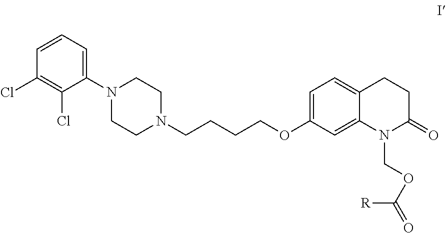

or a pharmaceutically acceptable salt thereof. R represents optionally substituted or unsubstituted alkyl group. The process comprises reacting a compound of formula III:

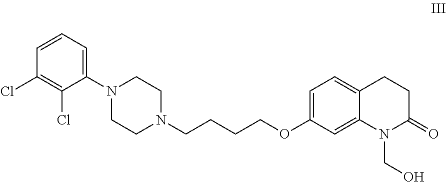 [SMILES: O=C1CCc2ccc(OCCCCN3CCN(c4cccc(Cl)c4Cl)CC3)cc2N1CO]

with a carboxylic acid in the presence of a coupling reagent and a first organic solvent to obtain the compound of formula I′. The carboxylic acid is preferably lauric acid. The coupling reagent is preferably selected from the group consisting of N, N′-dicyclohexylcarbodiimide (DCC), N, N′-diisopropylcarbodiimide (DIC), 1-ethyl-3-(3-dimethylaminopropyl)carbodiimide (EDC, EDAC or EDCI), and combinations thereof, and more preferably N, N′-dicyclohexylcarbodiimide (DCC). The first organic solvent is preferably toluene, DCM, or a combination thereof.

The process may further comprise reacting arpiprazole of formula II:

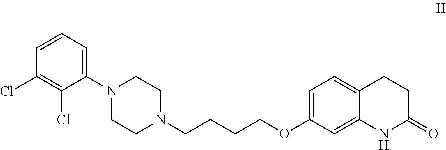 [SMILES: O=C1CCc2ccc(OCCCCN3CCN(c4cccc(Cl)c4Cl)CC3)cc2N1]

with an aldehyde in the presence of a base selected from the group consisting of tetra-n-butylammonium fluoride (TBAF), sodium carbonate monohydrate, and a combination thereof to provide the compound of formula III. The base is preferably tetra-n-butylammonium fluoride (TBAF). The aldehyde is preferably 37 wt. % aqueous solution of formaldehyde or paraformaldehyde, and more preferably 37 wt. % aqueous solution of formaldehyde.

The process may further comprise purifying the compound of formula I′ with a second organic solvent. The second organic solvent is preferably IPA. The compound of formula I′ is preferably arpiprazole lauroxil.

### DETAILED DESCRIPTION OF PREFERRED EMBODIMENTS OF THE INVENTION

The present application provides a process for preparing arpiprazole lauroxil.

In accordance with one embodiment, the present application provides a process for preparing a compound of formula I′:

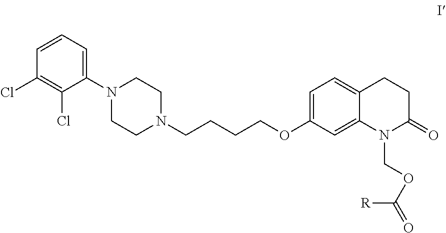

or a pharmaceutically acceptable salt thereof. R represents optionally substituted or unsubstituted alkyl group. The pharmaceutically acceptable salt may be hydrochloride, trifluroacetate, p-toluenesulfate sulfate, phosphate, acetate, benzoate, citrate, chiral dicarboxylic acid salts, and the like, preferably citrate. The optionally substituted alkyl group may be optionally substituted C₄-C₂₀, preferably C₈₋₁₄ alkyl group. The alkyl group may be substituted by hydrogen, halogen, amine, aliphatic, substituted aliphatic, aryl, or substituted aryl, preferably an aliphatic or substituted aliphatic group. The process may preferably comprise the following steps:
a) reacting the arpiprazole of formula II:

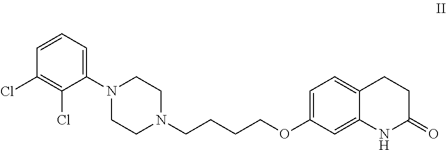 [SMILES: O=C1CCc2ccc(OCCCCN3CCN(c4cccc(Cl)c4Cl)CC3)cc2N1]

with an aldehyde in the presence of a base selected from the group consisting of tetra-n-butylammonium fluoride (TBAF), sodium carbonate monohydrate, and combination thereof to provide the compound of formula III:

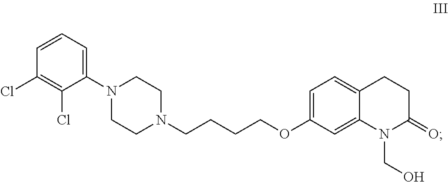 [SMILES: O=C1CCc2ccc(OCCCCN3CCN(c4cccc(Cl)c4Cl)CC3)cc2N1CO]

and
b) reacting the compound of formula III with a carboxylic acid in the presence of a coupling reagent and an organic solvent to obtain the compound of formula I′.

In some embodiments, the base used in step a) is tetra-n-butylammonium fluoride (TBAF). In other embodiments, the aldehyde used in step a) is preferably 37 wt. % aqueous solution of formaldehyde or paraformaldehyde. In a particular embodiment, the aldehyde is 37% aqueous solution of formaldehyde. The reacting step a) may be conducted under any appropriate conditions, for example, at a temperature from 20 to 50, preferably 20 to 45° C. for 20 to 72 hours, preferably 20 to 48 hours. The carboxlic acid may be C₈- to C-₁₄ acid. In certain embodiment, the carboxylic acid used in step b) is lauric acid. The organic solvent of step b) may be any organic solvent suitable for the reaction involved in step b), preferably toluene. In another embodiment, the process further comprises step c) to purify the compound of formula I with an organic solvent, such as IPA. The reacting step b) may be conducted under any appropriate conditions, for example, at a temperate from 20 to 50, preferably 20 to 30° C. for 2 to 6 hours, preferably 3 to 5 hours. The coupling reagent may be selected from N, N′-dicyclohexylcarbodiimide (DCC), N, N′-diisopropylcarbodiimide (DIC) or 1-Ethyl-3-(3-dimethylaminopropyl)carbodiimide (EDC, EDAC or EDCI), and more preferably N, N′-dicyclohexylcarbodiimide (DCC).

In accordance with yet another embodiment, the present invention provides a process for preparing arpiprazole lauroxil of formula I:

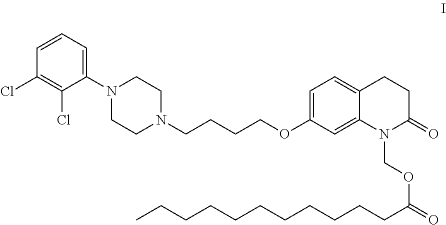 [SMILES: CCCCCCCCCCCC(=O)OCN1C(=O)CCc2ccc(OCCCCN3CCN(c4cccc(Cl)c4Cl)CC3)cc21]

or a pharmaceutically acceptable salt thereof, comprising the following steps of:
a) reacting the arpiprazole of formula II:

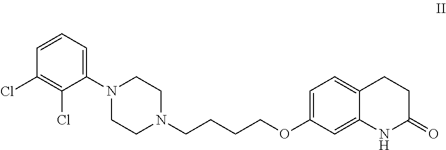 [SMILES: O=C1CCc2ccc(OCCCCN3CCN(c4cccc(Cl)c4Cl)CC3)cc2N1]

with an aldehyde in the presence of a base selected from the group consisting of tetra-n-butylammonium fluoride (TBAF), sodium carbonate monohydrate, and a combination thereof to provide the compound of formula III

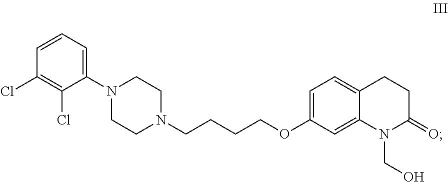 [SMILES: O=C1CCc2ccc(OCCCCN3CCN(c4cccc(Cl)c4Cl)CC3)cc2N1CO]

b) reacting the compound of formula III with lauric acid in the presence of a coupling reagent selected from N, N′-dicyclohexylcarbodiimide (DCC), N, N′-diisopropylcarbodiimide (DIC) or 1-Ethyl-3-(3-dimethylaminopropyl)carbodiimide (EDC, EDAC or EDCI), and an organic solvent to obtain arpiprazole lauroxil; and
c) purifying the compound of formula I with IPA.

In some embodiments, the base is tetra-n-butylammonium fluoride (TBAF). In other embodiments, the aldehyde used in step a) is 37% aqueous solution of formaldehyde or paraformaldehyde. In a particular embodiment, the aldehyde is 37% aqueous solution of formaldehyde. In certain embodiment, the organic solvent of step b) is toluene or DCM. The coupling reagent is preferably N′-dicyclohexylcarbodiimide (DCC).

### EXAMPLES

The following examples are provided to further illustrate, but not to limit this invention.

### Example 1: Preparation of M1

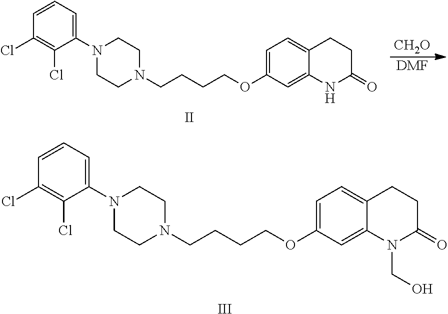

Aripiprazole (60 g, 133.81 mmol, 1.0 equiv.) and DMF (600 mL, 10 vol.) were added to a suitable reactor equipped with a mechanical stirrer and a thermometer at 20-30° C. and stirred for achieving a homogeneous solution. A 37% aqueous solution of formaldehyde (210 mL, 3.5 vol.) was slowly added at this temperature. A 1 M THF solution of TBAF (13.38 mL, 0.1 equiv.) was added to the mixture at 20-30° C. Then the reaction mixture was stirred at 20-30° C., followed by heating to 40-45° C. After the reaction was completed as determined by HPLC analysis, the resulting solution was cooled to 20-30° C. and stirred. Then the mixture was filtered and the filtered cake was washed with water (60 mL, 1 vol.) for two times. The wet cake was suction dried at 30° C. with N₂ purge to give 7-{4-[4-(2, 3-dichlorophenyl)-piperazin-1-yl]butoxy}-1-(hydroxymethyl)-3, 4-dihydroquinolin-2-one (N-hydroxymethyl aripiprazole, M1) as white solid (57.62 g, containing 6.5% Aripiprazole, 83.6% yield based on M1).

### Example 2: Preparation of Aripiprazole Lauroxil

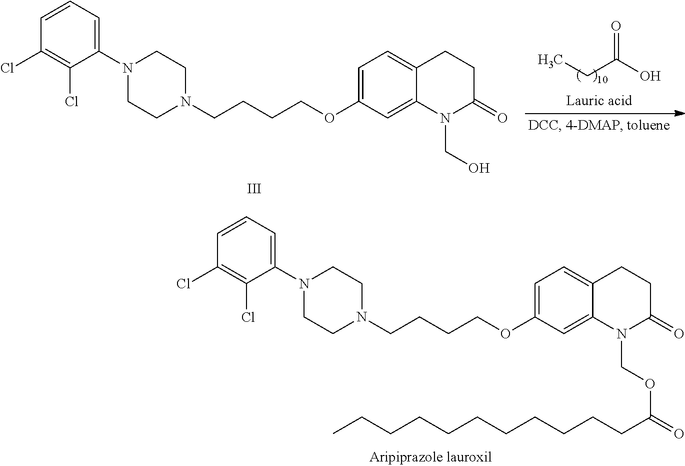

N-hydroxymethyl aripiprazole (M1) (50 g, 104.5 mmol, 1.0 equiv.) and dry toluene (500 mL, 10 vol.) were added to a suitable reactor equipped with a mechanical stirrer and a thermometer at 20-30° C. and stirred for about 5 min. Lauric acid (37.69 g, 188.1 mmol, 1.8 equiv.) and 4-DMAP (2.55 g, 20.9 mmol, 0.2 equiv.) were added to the mixture at this temperature. Dry toluene (100 mL, 2 vol.) was added to rinse the reactor. A solution of DCC (43.13 g, 209 mmol, 2.0 equiv.) in dry toluene (150 mL, 3 vol.) was slowly added to the mixture at this temperature. Then the reaction mixture was stirred at 20-30° C. for 4 hr. After the reaction was completed as determined by HPLC analysis, the solution was cooled to 0-10° C. (target 5° C.). 0.1 N HCl_{(aq)} (250 mL, 5 vol.) was slowly added to the mixture while maintaining temperature at NMT 10° C. The mixture was stirred at 0-10° C. for 30 min. Then the mixture was filtered, and the filtered cake was washed with toluene (150 mL, 3 vol.). 10% NaCl_{(aq)} (150 mL, 3 vol.) was added to the combined filtrate and washing. Then the combined filtrate and washing were subjected to phase separation. The separated organic portion was saved, and the separated aqueous portion was discarded. Water (250 mL, 5 vol.) was added to the reserved organic portion at 20-30° C. After the mixture was stirred for about 5 min, the stirring was stopped to affect phase separation. The separated organic portion was saved, and the separated aqueous portion was discarded.

The reserved organic portion was concentrated at 20-30° C. under 80-100 torr till volume reached about 5 vol. After more IPA (250 mL, 5 vol.) was added, the mixture was heated to 60-65° C. and stirred at this temperature for 10 min. Then the resulting reaction mixture was slowly cooled to 41-42° C. and stirred at this temperature for 1 hr. Then the mixture was cooled to 20-30° C. and stirred for 1 hr. The resulting mixture was cooled to 0-10° C. and stirred for 2 hr. The mixture was filtered, and the filtered cake was washed with pre-cooled IPA (50 mL, 1 vol.) for two times. The wet cake was suction dried at 30° C. with N₂ purge for 16 hr to give [7-{4-[4-(2, 3-dichlorophenyl)-piperazin-1-yl]butoxy}-2-oxo-3,4-dihydro-2H-quinolin-1-yl] methyl dodecanoate (crude API) as white solid (63.084 g, 91.4% yield based on API).

### Example 3: Purification of Aripiprazole Lauroxil

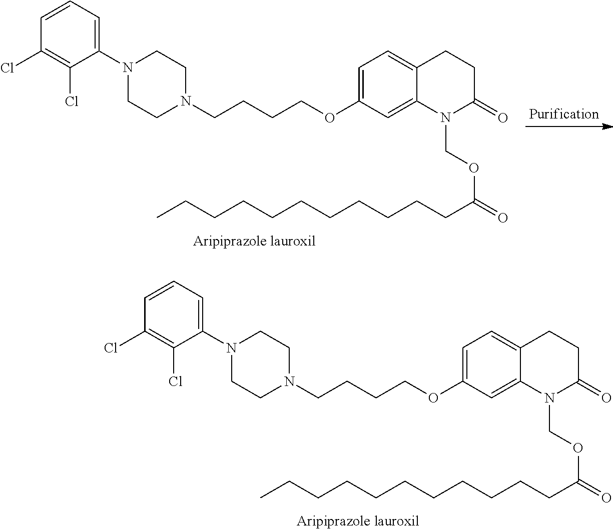 [SMILES: CCCCCCCCCCCC(=O)OCN1C(=O)CCc2ccc(OCCCCN3CCN(c4cccc(Cl)c4Cl)CC3)cc21 ; CCCCCCCCCCCC(=O)OCN1C(=O)CCc2ccc(OCCCCN3CCN(c4cccc(Cl)c4Cl)CC3)cc21]

[7-{4-[4-(2, 3-dichlorophenyl)-piperazin-1-yl]butoxy}-2-oxo-3,4-dihydro-2H-quinolin-1-yl]methyl dodecanoate (crude API) (62 g, 93.84 mmol, 1.0 equiv.) and IPA (620 mL, 10 vol.) were added to a suitable reactor equipped with a mechanical stirrer and a thermometer at 20-30° C. Reaction mixture was heated to 60-65° C. achieving a homogeneous solution and stirred for about 10 min. Then the resulting reaction mixture was slowly cooled to 41-42° C. and stirred at this temperature for 1 hr. The mixture was slowly cooled to 20-30° C. and stirred at this temperature for 1 hr. The resulting mixture was cooled to 0-10° C. and stirred for 2 hr. Then the mixture was filtered, and the filtered cake was washed with pre-cooled IPA (62 mL, 1 vol.) for two times. The wet cake was suction dried under N_{2(g)} purge to provide give [7-{4-[4-(2, 3-dichlorophenyl)-piperazin-1-yl]butoxy}-2-oxo-3,4-dihydro-2H-quinolin-1-yl] methyl dodecanoate (pure API) as white solid (59.59 g, 96.1% yield based on crude API, purity: 99.64%).

What is claimed is:

1. A process for preparing the compound of formula I′: 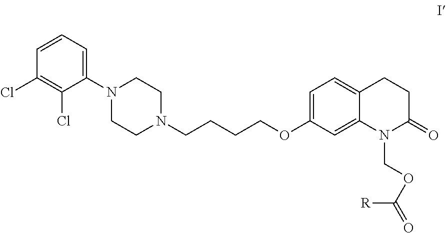
or a pharmaceutically acceptable salt thereof, wherein R represents an optionally substituted alkyl group, and the process comprises reacting a compound of formula III: 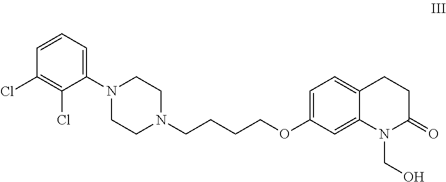 [SMILES: O=C1CCc2ccc(OCCCCN3CCN(c4cccc(Cl)c4Cl)CC3)cc2N1CO]
with a carboxylic acid in the presence of a coupling reagent and a first organic solvent to obtain the compound of formula I′.

2. The process of claim 1 further comprising reacting arpiprazole of formula II: 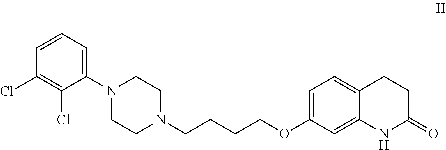 [SMILES: O=C1CCc2ccc(OCCCCN3CCN(c4cccc(Cl)c4Cl)CC3)cc2N1]
with an aldehyde in the presence of a base selected from the group consisting of tetra-n-butylammonium fluoride (TBAF), sodium carbonate monohydrate, and a combination thereof to provide the compound of formula III.

3. The process according to claim 2, wherein the base is tetra-n-butylammonium fluoride (TBAF).

4. The process according to claim 2, wherein the aldehyde is 37 wt. % aqueous solution of formaldehyde or paraformaldehyde.

5. The process according to claim 2, wherein the aldehyde is 37 wt. % aqueous solution of formaldehyde.

6. The process according to claim 1, wherein the carboxylic acid is lauric acid.

7. The process according to claim 1, wherein the coupling reagent is selected from the group consisting of N, N′-dicyclohexylcarbodiimide (DCC), N, N′-diisopropylcarbodiimide (DIC), 1-ethyl-3-(3-dimethylaminopropyl)carbodiimide (EDC, EDAC or EDCI), and combinations thereof.

8. The process according to claim 7, wherein the coupling reagent is N, N′-dicyclohexylcarbodiimide (DCC).

9. The process according to claim 1, wherein the first organic solvent is toluene, DCM, or combination thereof.

10. The process according to claim 1 further comprising purifying the compound of formula I′ with a second organic solvent.

11. The process according to claim 10, wherein the second organic solvent is Isopropyl alcohol (IPA).

12. The process according to claim 1, wherein the compound of formula I′ is arpiprazole lauroxil.

13. A process for preparing arpiprazole lauroxil of formula I: 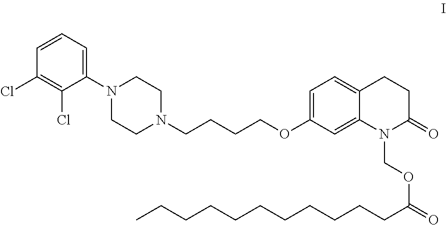 [SMILES: CCCCCCCCCCCC(=O)OCN1C(=O)CCc2ccc(OCCCCN3CCN(c4cccc(Cl)c4Cl)CC3)cc21]
or a pharmaceutically acceptable salt thereof comprising:
a) reacting arpiprazole of formula II: 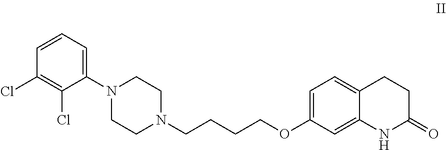 [SMILES: O=C1CCc2ccc(OCCCCN3CCN(c4cccc(Cl)c4Cl)CC3)cc2N1]
with a 37 wt. % aqueous solution of formaldehyde in the presence of tetra-n-butylammonium fluoride (TBAF) to provide the compound of formula III: 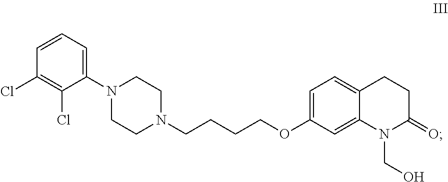 [SMILES: O=C1CCc2ccc(OCCCCN3CCN(c4cccc(Cl)c4Cl)CC3)cc2N1CO]
b) reacting the compound of formula III with lauric acid in the presence of a coupling reagent selected from the group consisting of N, N′-dicyclohexylcarbodiimide (DCC), N, N′-diisopropylcarbodiimide (DIC), 1-ethyl-3-(3-dimethylaminopropyl)carbodiimide (EDC, EDAC or EDCI), and combinations thereof, and an organic solvent selected from the group consisting of toluene, DCM, and a combination thereof, to obtain arpiprazole lauroxil of formula I; and
c) purifying the arpiprazole lauroxil of formula I with IPA.
